(19) United States
(12) Patent Application Publication
(10) Pub. No.: US 2016/0151171 A1
(43) Pub. Date: Jun. 2, 2016

(54) MODULAR INTERVERTEBRAL CAGE SYSTEM

(71) Applicant: SPINAL USA, INC., Parsippany, NJ (US)

(72) Inventors: Scott E. MOZELESKI, Hoboken, NJ (US); Michael N. CAMPBELL, Elmwood Park, NJ (US)

(21) Appl. No.: 14/905,437

(22) PCT Filed: Jul. 16, 2014
(86) PCT No.: PCT/US2014/046804

§ 371 Date: Jan. 15, 2016

Related U.S. Application Data
(60) Provisional application No. 61/847,506, filed on Jul. 17, 2013.

(51) Int. Cl. A61F 2/44

(52) U.S. Cl. CPC A61F 2/4455; A61F 2/442; A61F 2220/0016

(57) ABSTRACT

Devices and methods are provided for spinal disc replacement. A plate can be configured to couple to an intervertebral cage disposed in an intervertebral space. The plate can be modular. For example, the plate can have a first receiving portion angled relative to a second receiving portion. The first receiving portion receives a first bone screw or bone blade to engage a first vertebra, and the second receiving portion receives a second bone screw or bone blade to engage a second vertebra. A retention member can be coupled to the plate. The retention member is configured to block first and second bone screws or bone blades from backing out of the plate.

### CROSS-REFERENCE TO RELATED APPLICATIONS

This application claims priority benefit of U.S. Provisional Application No. 61/847,506, entitled “MODULAR INTERVERTEBRAL CAGE SYSTEM,” filed Jul. 17, 2013, the entirety of which is hereby incorporated by reference.

### TECHNICAL FIELD

The present application relates to systems, devices, and methods for spinal surgeries. In particular, the present application relates to systems, devices, and methods for disc replacement surgeries.

### DESCRIPTION OF THE RELATED TECHNOLOGY

The spine relies on intervertebral spinal discs in between adjacent vertebrae to serve as mechanical cushions and transmit compressive loads. Spinal discs are composed of an outer annulus fibrosus that surrounds an inner nucleus pulposus. The annulus fibrosus is composed of laminae of fibrous tissue and fibrocartilage, while the nucleus pulposus is composed of water, chondrocytes, collagen fibrils and proteoglycan aggrecans that have hyaluronic long chains. The nucleus pulposus functions to distribute hydraulic pressure in all directions within each disc under compressive loads.

The nucleus pulposus, which begins early in life as eighty percent water, slowly dessicates with age. This causes the spinal disc to lose its cushioning ability and ability to bear loads, resulting in pain in the back and lower extremities. To resolve these problems, the degenerated nucleus may be removed and replaced. In some other cases, the nucleus may be removed and the vertebrae may be fused together in a spinal fusion procedure, which may include implanting an intervertebral cage and/or bone growth material to facilitate fusion of the vertebrae.

During vertebral disc replacement surgery, it is commonplace to insert an intervertebral spacer between two adjacent vertebrae in the place of a ruptured or diseased disc. Such intervertebral spacers can include, but are not limited to, bone grafts, peek cages, titanium cages, stainless steel cages, bioresorbable cages, and the like. In some circumstances, bone screws are passed through the intervertebral cage and into the adjacent vertebrae in order to anchor the cage in place.

### SUMMARY

The systems, methods and devices described herein have innovative aspects, no single one of which is indispensable or solely responsible for their desirable attributes. Without limiting the scope of the claims, some of the advantageous features will now be summarized.

One aspect of the present invention is the realization that the anatomy of some patients' spines is prohibitive to utilizing existing intervertebral cage designs and fixation means. Thus, there exists a need for a modular cage system which does not suffer from the deficiencies of conventional intervertebral cages.

One non-limiting embodiment of the present invention includes a plate configured to couple to an intervertebral cage, the intervertebral cage configured and arranged to be positioned in an intervertebral space between a first vertebra and a second vertebra, wherein the plate comprises an upper surface configured to engage the first vertebra and a lower surface opposite the upper surface, the lower surface configured to engage the second vertebra, wherein the plate comprises a posterior face configured to face the intervertebral space and abut an anterior face of the intervertebral cage and an anterior face configured to face outward from the intervertebral space; wherein the plate comprises a first receiving portion and a second receiving portion, the first receiving portion angled relative to the second receiving portion, the first receiving portion configured to receive a first bone fixation member, the first bone fixation member comprising either a bone screw or a bone blade, the first bone fixation member configured to engage a first vertebra, the second receiving portion configured to receive a second bone fixation member, the second bone fixation member comprising either a bone screw or a bone blade, the second bone fixation member configured to engage a second vertebra, the first and second receiving portions each configured to optionally receive either a bone screw or a bone blade.

In another embodiment, the first and second receiving portions each comprise at least one guidance rail configured to guide a bone blade into a vertebra.

In another embodiment, the first and second receiving portions each comprise a through bore and a head engaging portion, each through bore configured to receive the bone engaging portion of the bone fixation member, each head engaging portion configured to block the head of the bone fixation member from passing completely through the receiving portion.

In another embodiment the through bore of the first and second receiving portions each comprise at least one guidance rail, wherein each guidance rail comprises a recess formed therein a wall of each through bore, each guidance rail configured to accept a portion of the bone blade and guide the bone blade into a vertebra as it is passed through the receiving portion.

In another embodiment, each receiving portion is configured to receive a bone blade, wherein the bone blade comprises a head and a bone engaging portion, wherein the bone engaging portion of the bone blade comprises a curve along its length, wherein the bone engaging portion of the bone blade comprises a major rib, wherein the bone blade is configured to engage a vertebra via translation without rotation of the bone blade, wherein the each guidance rail is configured to accept the major rib of the bone blade and guide the bone blade into a vertebra.

In another embodiment, the bone engaging portion of the bone blade comprises a minor rib arranged perpendicular to the major rib.

In another embodiment, each through bore of each receiving portion comprises a central axis, and wherein each of the guidance rails are arranged substantially parallel to a central axis of the through bore.

In another embodiment, an intervertebral cage configured and arranged to be positioned in an intervertebral space between a first vertebra and a second vertebra, the intervertebral cage comprising an upper surface and a lower surface opposite the upper surface, the intervertebral cage comprising a posterior face and an anterior face opposite said posterior face, the intervertebral cage comprising at least two sidewalls; a plate configured to couple to the intervertebral cage, the intervertebral cage configured and arranged to be positioned in the intervertebral space, wherein the plate comprises an upper surface configured to engage the first vertebra and a lower surface opposite the upper surface, the lower surface configured to engage a second vertebra, wherein the plate comprises a posterior face configured to face the intervertebral space and abut an anterior face of the intervertebral cage and an anterior face configured to face outward from the intervertebral space; at least two bone fixation members adapted for insertion through the receiving portions of the plate and configured for anchoring the plate in an intervertebral space between two vertebrae of a patient, the bone fixation members comprising at least a first bone fixation member and a second bone fixation member, the first bone fixation member comprising either a bone screw or a bone blade and the second bone fixation member comprising either a bone screw or a bone blade, the first bone fixation member configured to engage the first vertebra, the second bone fixation member configured to engage the second vertebra; wherein the intervertebral cage comprises a first cage bore formed therethrough the anterior face and the upper surface configured to receive the first bone fixation member and a second cage bore formed therethrough the anterior face and the lower surface configured to receive the second bone fixation member; wherein the plate comprises a first receiving portion and a second receiving portion, the first receiving portion angled relative to the second receiving portion, the first receiving portion configured to receive the first bone fixation member, the second receiving portion configured to receive a second bone fixation member, the first and second receiving portions each configured to optionally receive either a bone screw or a bone blade; wherein the plate comprises a plate coupling portion configured to couple the plate to the intervertebral cage; and wherein the intervertebral cage comprises a cage coupling portion configured to couple the cage to the plate.

In another embodiment, the intervertebral cage is constructed of PEEK and the plate is constructed of titanium.

In another embodiment, the system includes a retention member coupled to the plate, the retention member configured to block first and second bone fixation members from backing out of the plate; wherein the retention member comprises a shaft portion and a blocking portion, the shaft portion configured to engage and couple the retention member to the plate, the blocking portion configured to engage the first and second bone fixation members; wherein the retention member is configured to rotate between a locked position and an unlocked positions such that when in an unlocked position, the first and second bone fixation members can be installed through the first and second receiving portions of the plate, and in a locked position, the first and second bone fixation members are locked in place.

In another embodiment, the blocking portion comprises at least a first clearance side and a first interference side, and a second clearance side and a second interference side, wherein the first and second clearance sides each comprise a recess formed therein the blocking portion of the retention member, providing room for the first and second bone fixation members to enter the first and second receiving portions of the plate, wherein the first and second interference sides include first and second engaging portions respectively, the first engaging portion configured to engage the head of the first bone fixation member and the second engaging portion configured to engage the head of the second bone fixation member when the retention member is rotated from an unlocked position to a locked position.

In another embodiment, the retention member is configured such that interference fit exists between the blocking portion of the retention member and the heads of the first and second bone fixation members when the retention member is in a locked position.

In another embodiment, the first and second receiving portions each comprise at least one guidance rail configured to guide a bone fixation member comprising a bone blade into a vertebra, the first and second receiving portions each comprise a through bore and a head engaging portion, each through bore configured to receive the bone engaging portion of the bone fixation member, each head engaging portion configured to block the head of the bone fixation member from passing completely through the receiving portion, wherein the through bore of the first and second receiving portions each comprise at least one guidance rail, wherein each guidance rail comprises a recess formed therein a wall of each through bor, each guidance rail configured to accept a portion of the bone blade and guide the bone blade into a vertebra as it is passed through the receiving portion, each receiving portion is configured to receive a bone blade, wherein the bone blade comprises a head and a bone engaging portion, wherein the bone engaging portion of the bone blade comprises a curve along its length, wherein the bone engaging portion of the bone blade comprises a major rib, wherein the bone blade is configured to engage a vertebra via translation without rotation of the bone blade, wherein the each guidance rail is configured to accept the major rib of the bone blade and guide the bone blade into a vertebra, and wherein each through bore of each receiving portion comprises a central axis, and wherein each of the guidance rails are arranged substantially parallel to the central axis of the through bore.

In another embodiment, the cage coupling portion of the intervertebral cage comprises at least one channel configured to receive a portion of the plate, and wherein the plate coupling portion of the plate comprises at least one clasp extending outwards from the posterior face of the plate, the at least one clasp configured to enter the channel of the intervertebral cage and couple the plate to the intervertebral cage.

In another embodiment, the intervertebral cage comprises at least one chamfer configured to deflect the at least one clasp, wherein the intervertebral cage comprises at least one recess formed in the at least one channel, the recess configured to receive a portion of the at least one clasp, wherein the at least one clasp comprises a protrusion configured to engage the recess of the intervertebral cage, and wherein the at least one clasp is configured to deflect when coupling the intervertebral cage and to return to toward the undeflected position when the protrusion engages the recess of the intervertebral cage and couples the plate to the intervertebral plate.

In another embodiment, the plate comprises a plate height defined by the distance between the upper surface and the lower surface of the plate, wherein the intervertebral cage comprises an anterior cage height defined by the distance between the upper surface and the lower surface of the cage measured at an anterior face of the intervertebral cage, wherein the plate height is substantially the same as the anterior cage height.

In another embodiment, A method for installing a spinal implant system includes positioning an intervertebral cage in an intervertebral space between a first vertebra and a second vertebra; positioning a plate at least partially in the intervertebral space and coupling the plate to the intervertebral cage; wherein coupling the plate to the intervertebral cage comprises forcing the plate against the anterior face of the intervertebral cage; installing a first bone fixation element through a first receiving portion of the plate and into a first vertebra, the first vertebra located above the intervertebral cage; installing a second bone fixation element through a first receiving portion of the plate and into a second vertebra, the second vertebra located below the intervertebral cage; wherein at least one of the first bone fixation element and second bone fixation element comprises a bone blade; rotating a retention member 90 degrees to lock the first and second bone fixation elements in place.

In another embodiment, installation of the bone blade comprises aligning a major rib of the bone blade in a guidance rail located in the first or second receiving portion and exerting a force substantially parallel to a center axis of the bone blade in the posterior direction causing the bone blade to enter the first or second vertebra.

In another embodiment, the bone blade comprises a curve along a bone engaging portion of the bone blade.

In another embodiment, rotating a retention member comprises creating an interference fit between the retention member and the first and second bone fixation elements, locking the first and second bone fixation elements in place.

In another embodiment, a method of removing a bone blade which is implanted into a intervertebral cage and a vertebra includes maneuvering a bone blade extraction tool towards the bone blade, rotating the bone blade extraction tool until at least one extraction lobe of the bone blade extraction tool aligns with at least one lobe recess of a tool receiving portion of the bone blade, inserting the bone blade extraction tool into the tool receiving portion of the bone blade until the at least one extraction lobe reaches an undercut channel of the tool receiving portion, rotating the bone blade extraction tool until the at least one extraction lobe locks the bone blade extraction tool to the bone blade, and pulling the bone blade extraction tool away from the intervertebral cage.

In another embodiment, at least one extraction lobe locking the bone blade extraction tool to the bone blade includes the at least one extraction lobe achieving an interference fit between the at least one extraction lobe and the undercut channel.

In another embodiment, the undercut channel comprises a decreasing diameter configured to achieve an interference fit with the at least one extraction lobe of the bone blade extraction tool.

### BRIEF DESCRIPTION OF THE DRAWINGS

The above-mentioned aspects, as well as other features, aspects, and advantages of the present technology will now be described in connection with various embodiments, with reference to the accompanying drawings. The illustrated embodiments, however, are merely examples and are not intended to be limiting. Like reference numbers and designations in the various drawings indicate like elements.

FIG. 1 illustrates a perspective view of one embodiment of a modular cage system.

FIG. 2 illustrates a perspective view of one embodiment of an intervertebral cage.

FIG. 3 illustrates a perspective view of one embodiment of a plate.

FIG. 4 illustrates a top section view of the intervertebral cage of FIG. 2.

FIG. 5 illustrates a top view of the plate of FIG. 3.

FIG. 6A illustrates a front view of the modular cage system of FIG. 1.

FIG. 6B illustrates a side view of the modular cage system of FIG. 1.

FIG. 7A illustrates a perspective view of one embodiment of a bone blade.

FIG. 7B illustrates a side view of the bone blade of FIG. 7A.

FIG. 8 illustrates a perspective view of the intervertebral cage of FIG. 2.

FIG. 9 illustrates a top section view of the plate of FIG. 3 including one embodiment of a retention member in a locked position.

FIG. 10A illustrates a perspective view of one embodiment of a retention member.

FIG. 10B illustrates a top view of the retention member of FIG. 10A.

FIG. 10C illustrates a perspective view of the retention member of FIG. 10A.

FIG. 10D illustrates a side view of the retention member of FIG. 10A.

FIG. 11 illustrates a partial section view of the bone blade of FIG. 7A.

FIG. 12 illustrates a partial section view of one embodiment of a bone blade insertion tool engaged to the bone blade of FIG. 7A.

FIG. 13 illustrates a partial section view of the bone blade insertion tool of FIG. 12 engaged to the bone blade of FIG. 7A and approaching the delivery site.

FIG. 14 illustrates a partial section view of the bone blade of FIG. 7A fully installed in the plate and engaged by the bone blade insertion tool of FIG. 12.

FIG. 15 illustrates a front view of the modular cage system of FIG. 1 including one embodiment of a bone blade extraction tool.

FIG. 16 illustrates a partial section view of the bone blade of FIG. 7A.

FIG. 17 illustrates a partial section view of the bone blade of FIG. 7A fully installed in the plate and engaged by the bone blade extraction tool of FIG. 15.

### DETAILED DESCRIPTION

In the following detailed description, reference is made to the accompanying drawings, which form a part of the present disclosure. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and form part of this disclosure. For example, a system or device may be implemented or a method may be practiced using any number of the aspects set forth herein. In addition, such a system or device may be implemented or such a method may be practiced using other structure, functionality, or structure and functionality in addition to or other than one or more of the aspects set forth herein. Alterations and further modifications of the inventive features illustrated herein, and additional applications of the principles of the inventions as illustrated herein, which would occur to one skilled in the relevant art and having possession of this disclosure, are to be considered within the scope of the invention.

Descriptions of unnecessary parts or elements may be omitted for clarity and conciseness, and like reference numerals refer to like elements throughout. In the drawings, the size and thickness of layers and regions may be exaggerated for clarity and convenience.

Features of the present disclosure will become more fully apparent from the following description and appended claims, taken in conjunction with the accompanying drawings. It will be understood these drawings depict only certain embodiments in accordance with the disclosure and, therefore, are not to be considered limiting of its scope; the disclosure will be described with additional specificity and detail through use of the accompanying drawings. An apparatus, system or method according to some of the described embodiments can have several aspects, no single one of which necessarily is solely responsible for the desirable attributes of the apparatus, system or method. After considering this discussion, and particularly after reading the section entitled “Detailed Description” one will understand how illustrated features serve to explain certain principles of the present disclosure.

Embodiments described herein generally relate to systems, devices, and methods for spinal surgeries. More specifically, some embodiments relate to systems, devices, and methods for spinal fusion surgeries.

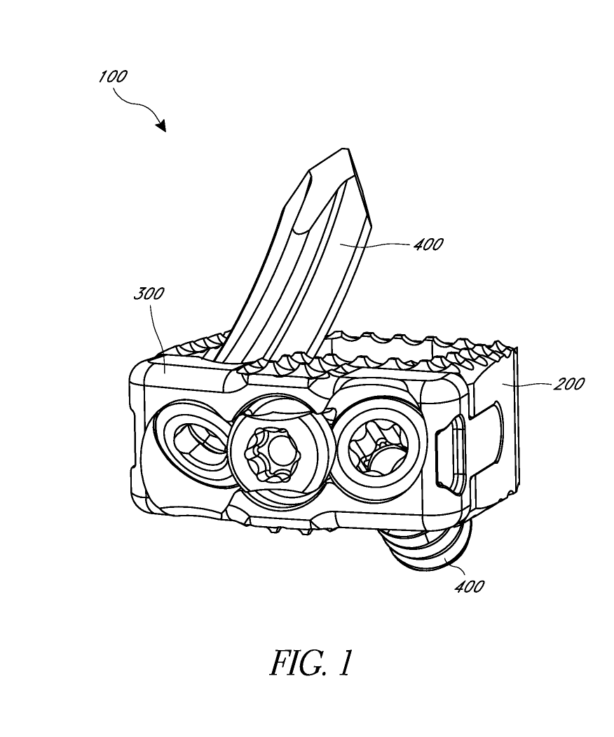

FIG. 1 illustrates a perspective view of one embodiment of a modular cage system 100. In some embodiments, the modular cage system 100 can include an intervertebral cage 200 configured and arranged to be positioned in an intervertebral space between two vertebrae of a patient during spinal surgery. In some embodiments, the modular cage system 100 can include a plate 300 configured to couple to the intervertebral cage 200. In some embodiments, the plate 300 can be coupled to an intervertebral cage 200 before implantation of the intervertebral cage 200. In some embodiments, the plate 300 can be coupled to the intervertebral cage 200 after implantation of the intervertebral cage 200. In some embodiments, the intervertebral cage 200 and plate 300 can be formed integrally and comprise a single piece. One advantage to separate intervertebral cage 200 and plate 300 components is the ability to mix and match different configurations of plate 300 with different configurations of intervertebral cage 200. In addition, the modular construction of the modular cage system 100 can minimize inventory levels. In some embodiments, the intervertebral cage 200 and plate 300 can be made from different materials wherein the material for each component of the modular cage system 100 can be selected to optimally achieve the goal of each component. In some embodiments, the intervertebral cage 200 can be made from polyether ether ketone (PEEK). In some embodiments, the plate 300 can be constructed from a metal material. In some embodiments, the plate 300 can be constructed from titanium, which for example, offers advantages such as strength, robustness, and flexibility. In some embodiments, the intervertebral cage 200, plate 300, retention member 500, or additional portion of the modular cage system 100 can be made from biocompatible materials which may include, for example, metal, titanium, stainless steel, Nitinol, pyrolitic carbon, polymers, polyether ether ketone, silicone methylmethacrylate, or other biocompatible materials known in the art.

In some embodiments, the modular cage system 100 can include a plurality of bone fixation members 400 adapted to anchor the modular cage system 100 in an intervertebral space between two vertebrae of a patient. In some embodiments, the bone fixation members 400 can be configured to pass through the intervertebral cage 200. In some embodiments, the bone fixation members 400 can be configured to pass through the plate 300. In some embodiments, the bone fixation members 400 can be configured to engage the adjacent vertebrae.

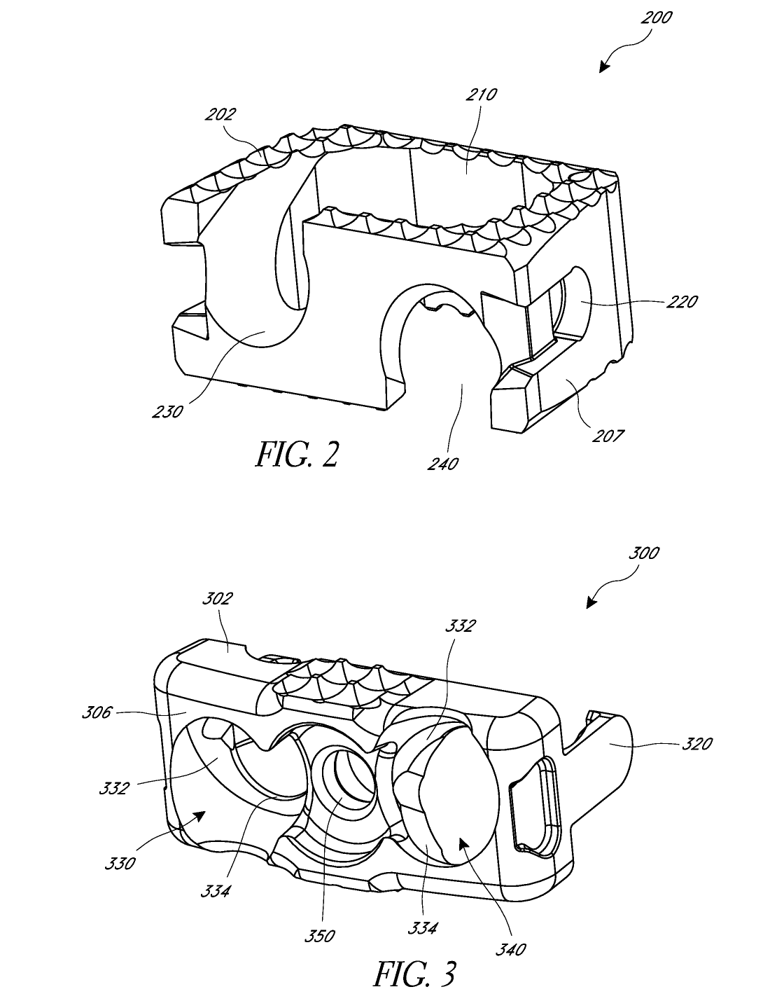

FIG. 2 illustrates a perspective view of one embodiment of an intervertebral cage 200. In some embodiments, the intervertebral cage 200 can include an upper surface 202. The upper surface 202 can be configured to engage a vertebra above the intervertebral cage 200. The intervertebral cage 200 can include a lower surface 204 opposite the upper surface 202. The lower surface 204 can be configured to engage a vertebra below the intervertebral cage 200. In some embodiments, the upper surface 202 and lower surface 204 can include a surface texture configured to retain the intervertebral cage 200 in the intervertebral space between two vertebrae and minimize movement of the vertebrae relative to the intervertebral cage 200. In some embodiments, the surface texture can be configured to promote bone ingrowth and fusion. In some embodiments, the surface texture can include a pattern of peaks and valleys. In some embodiments, the surface texture can include a plurality of pyramid-shaped bumps. In some embodiments, the bumps can be created using a radius cut. In some embodiments, the bumps can be slanted to limit migration of the intervertebral cage 200 in a particular direction. In some embodiments, the bumps can be slanted towards the posterior face 208 of the intervertebral cage 200. In some embodiments, the bumps can be slanted towards the anterior face 206 of the intervertebral cage 200.

In some embodiments, the intervertebral cage 200 can include a graft window 210. The graft window 210 can be formed through the upper surface 202 and lower surface 204 of the intervertebral cage 200. The graft window 210 can be configured to promote bone ingrowth and fusion. In some embodiments, the graft window 210 can be filled or partially filled with bone graft material prior to, during, or after implantation. Bone graft material can include autologous, allograft, or synthetic materials which may include, for example, hydroxyapatite, tricalcium phosphate, bioglass, or calcium sulphate. In some embodiments, growth factors can be included in the graft window 210. In some embodiments, the intervertebral cage 200 can include at least one marker device configured to be viewed via fluoroscopy and aid in the positioning of the intervertebral cage 200.

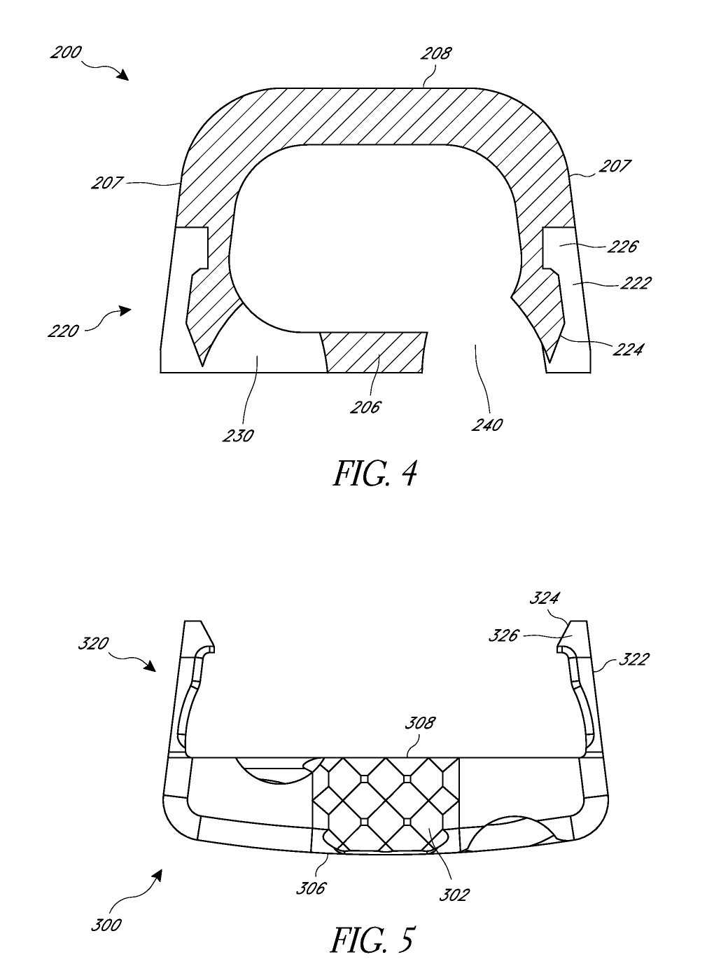

In some embodiments, the intervertebral cage 200 can include an anterior face 206 and a posterior face 208 (See FIG. 4) opposite the anterior face 206. The intervertebral cage 200 can include sidewalls 207 on each side of the intervertebral cage 200. In some embodiments, the anterior face 206 can be configured to abut the posterior face 308 of the plate 300. In some embodiments, intervertebral cage 200 can include a cage coupling portion 220 configured to couple the cage to the plate 300. In some embodiments, the sidewalls 207 of the intervertebral cage 200 can include a cage coupling portion 220 configured to couple the intervertebral cage 200 to the plate 300. In some embodiments, the anterior face 206 of the intervertebral cage 200 can include a cage coupling portion 220 configured to couple the cage to the plate 300. In some embodiments, the cage coupling portion 220 can be part of a different portion of the intervertebral cage 200, which may include for example, the upper surface 202, the lower surface 204, the graft window 210, etc.

In some embodiments, the intervertebral cage 200 can be configured to accept a plurality of bone fixation members 400. In some embodiments, the intervertebral cage 200 can be configured to allow a plurality of bone fixation members 400 to pass through the intervertebral cage 200. The intervertebral cage 200 can include a plurality of cage bores 230, 240 each configured to accept a bone fixation member 400. In some embodiments, the plurality of cage bores 230, 240 can include a first cage bore 230 and a second cage bore 240. In some embodiments, the first cage bore 230 can be configured to accept a first bone fixation member 400 wherein the first bone fixation member 400 is angled upwards and configured to engage a vertebra above the intervertebral cage 200. The first cage bore 230 can be formed therethrough the anterior face 206 and the upper surface 202 of the intervertebral cage 200. In some embodiments, the second cage bore 240 can be configured to accept a second bone fixation member 400 wherein the second bone fixation member 400 is angled downwards and configured to engage a vertebra below the intervertebral cage 200. The second cage bore 240 can be formed therethrough the anterior face 206 and the lower surface 204 of the intervertebral cage 200.

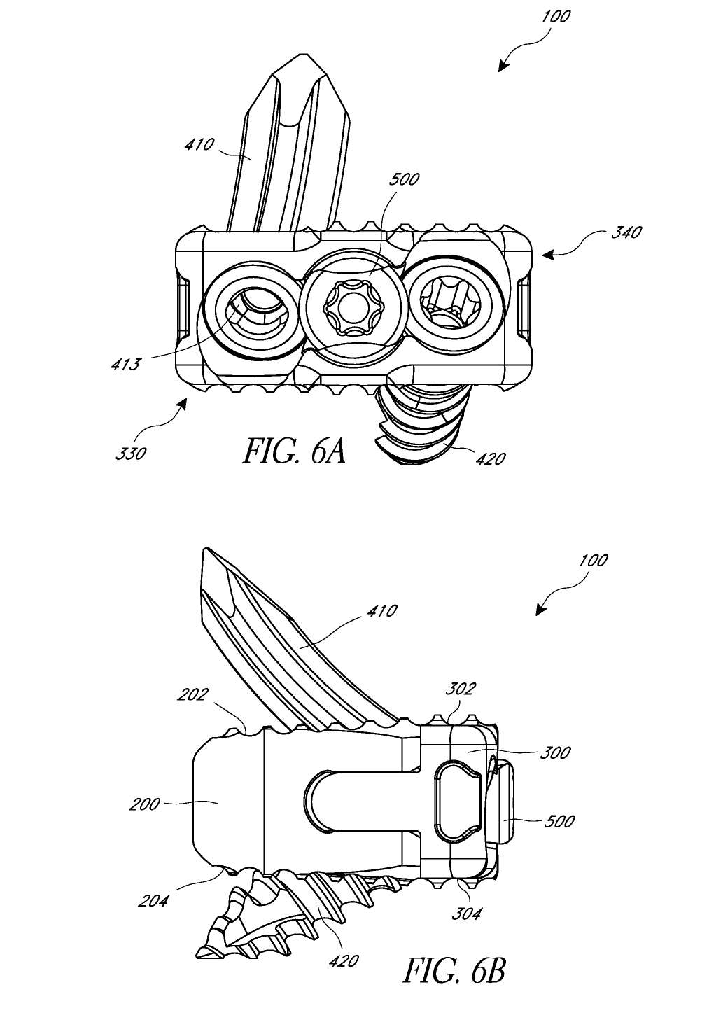

In some embodiments, the modular cage system 100 can be configured to angle the bone fixation member 400 between approximately 15 and 75 degrees relative to the modular cage system 100 as illustrated in FIG. 6B. In some embodiments, the modular cage system 100 can be configured to angle the bone fixation member 400 between approximately 20 and 60 degrees relative to the modular cage system 100. In some embodiments, the modular cage system 100 can be configured to angle the bone fixation member 400 between approximately 25 and 50 degrees relative to the modular cage system 100. In some embodiments, the modular cage system 100 can be configured to angle the bone fixation member 400 between approximately 25 and 40 degrees relative to the modular cage system 100. In some embodiments, the modular cage system 100 can be configured to angle the bone fixation member 400 between approximately 30 and 35 degrees relative to the modular cage system 100. In some embodiments, the modular cage system 100 can be configured to angle the bone fixation member 400 approximately 30 degrees relative to the modular cage system 100. In some embodiments, the angle of the bone fixation member 400 can be configured to account for the anatomy of a patient's spine.

In some embodiments, the upper surface 202 of the intervertebral cage 200 can be angled relative to the lower surface 204. In some embodiments, the upper surface 202 is further from the lower surface 204 near the anterior face 206 of the intervertebral cage 200 and the upper surface 202 is closer to the lower surface 204 near the posterior face 208 of the intervertebral cage 200. In some embodiments, the upper surface 202 is further from the lower surface 204 near the posterior face 208 of the intervertebral cage 200 and the upper surface 202 is closer to the lower surface 204 near the anterior face 206 of the intervertebral cage 200. In some embodiments, the upper surface 202 of the intervertebral cage 200 is substantially parallel to the lower surface 204.

FIG. 3 illustrates a perspective view of one embodiment of a plate 300. In some embodiments, the plate 300 can be configured to couple to the intervertebral cage 200. In some embodiments, the plate 300 can include an upper surface 302. The upper surface 302 can be configured to engage a vertebra above the plate 300. The plate 300 can include a lower surface 304 opposite the upper surface 302. The lower surface 304 can be configured to engage a vertebra below the plate 300. In some embodiments, the upper surface 302 and lower surface 304 of the plate 300 can include a surface texture configured to retain the plate 300 in the intervertebral space between two vertebrae as described above in relation to the intervertebral cage 200 and limit movement of the vertebrae relative to the plate 300.

In some embodiments, the plate 300 can include an anterior face 306 and a posterior face 308 opposite the anterior face 306. The posterior face 308 can be configured to face the intervertebral space. The anterior face 306 can be configured to face outwards from the intervertebral space. In some embodiments, the posterior face 308 can be configured to abut the anterior face 206 of the intervertebral cage 200. In some embodiments, the plate 300 can include a plate coupling portion 320 configured to couple the plate 300 to the intervertebral cage 200.

In some embodiments, the plate 300 can be configured to accept a plurality of bone fixation members 400. In some embodiments, the plate 300 can be configured to allow a plurality of bone fixation members 400 to pass through the plate 300. The plate 300 can include a plurality of receiving portions configured to accept bone fixation members 400. In some embodiments, the plurality of receiving portions can include a first receiving portion 330 and a second receiving portion 340. In some embodiments, the first receiving portion 330 can be configured to accept a first bone fixation member 400 wherein the first bone fixation member 400 is angled upwards and configured to engage a vertebra above the intervertebral cage 200. In some embodiments, the first receiving portion 330 can be formed therethrough the anterior face 306 and posterior face 308 of the plate 300. In some embodiments, the second receiving portion 340 can be configured to accept a second bone fixation member 400 wherein the second bone fixation member 400 is angled downwards and configured to engage a vertebra below the intervertebral cage 200. In some embodiments, the first receiving portion 330 can be formed therethrough the anterior face 306 and posterior face 308 of the plate 300.

In some embodiments, the first and second receiving portion 340s can include a through bore 334 and a head engaging portion 332. The through bore 334 can be configured to receive the bone engaging portion 414 of the bone fixation member 400. The head engaging portion 332 can be configured to block the head 412 of the bone fixation member 400 from passing completely through the receiving portion and retain the modular cage system 100 in the intervertebral space.

In some embodiments, the upper surface 302 of the plate 300 can be angled relative to the lower surface 304 of the plate 300. In some embodiments, the upper surface 302 is further from the lower surface 304 near the anterior face 306 of the plate 300 and the upper surface 302 is closer to the lower surface 304 near the posterior face 308 of the plate 300. In some embodiments, the upper surface 302 is further from the lower surface 304 near the posterior face 308 of the plate 300 and the upper surface 302 is closer to the lower surface 304 near the anterior face 306 of the plate 300. In some embodiments, the upper surface 302 of the plate 300 is substantially parallel to the lower surface 304. The intervertebral cage 200 can have an anterior cage height defined by the distance between the upper surface 202 of the intervertebral cage 200 and the lower surface 204 of the intervertebral cage 200 measured at an anterior face 206 of the intervertebral cage 200. The plate 300 can comprise a plate 300 height defined by the distance between the upper surface 302 of the plate 300 and the lower surface 304 of the plate 300. In some embodiments, the plate 300 height is configured to be substantially similar to the anterior cage height such that the upper surface 302 of the plate 300 lines up with the upper surface 202 of the intervertebral cage 200 and the lower surface 304 of the plate 300 lines up with the lower surface 204 of the intervertebral cage 200 near the anterior face 206 of the intervertebral cage 200.

FIG. 4 illustrates a top section view of the intervertebral cage 200 of FIG. 2. FIG. 5 illustrates a top view of the plate 300 of FIG. 3. In some embodiments, the cage coupling portion 220 of the intervertebral cage 200 can include at least one channel 222 configured to receive a portion of the plate 300. In some embodiments, the plate coupling portion 320 of the plate 300 can include at least one clasp 322 configured to enter the channel 222 of the intervertebral cage 200. In some embodiments, the channel 222 of the cage coupling portion 220 and the clasp 322 of the plate coupling portion 320 are configured such that the clasp 322 locks into place once forced into the channel. In some embodiments, the channel 222 of the cage coupling portion 220 and the clasp 322 of the plate coupling portion 320 are configured such that once the clasp 322 is locked into place, the plate 300 is coupled to the intervertebral plate 300. In some embodiments, the posterior face 308 of the plate 300 and the anterior face 206 of the intervertebral cage 200 are complementary such that when the plate 300 is coupled to the intervertebral cage 200, the plate 300 is restricted from moving relative to the intervertebral cage 200. In some embodiments, the clasp 322 can include an arm extending outward from the posterior face 308 of the plate 300 and extend into the intervertebral space. In some embodiments, the posterior face 308 of the plate 300 and the anterior face 206 of the intervertebral cage 200 can be substantially flat. In some embodiments, the posterior face 308 of the plate 300 and the anterior face 206 of the intervertebral cage 200 can incorporate complementary features, which may include for example, protrusions 326 and recesses 226, configured to limit relative movement between the intervertebral cage 200 and plate 300. In some embodiments, the clasp 322 can be configured to deflect during the coupling process and to return toward the undeflected position when locking into position in the channel 222. In some embodiments, the intervertebral cage 200 can include a chamfer 224 configured to deflect the clasp 322 outwards during the coupling process.

In some embodiments, the intervertebral cage 200 can include at least one recess 226 configured to accept a portion of the plate coupling portion 320 of the plate 300 and couple the plate 300 to the intervertebral cage 200. In some embodiments, the recess 226 can be formed in the channel 222. In some embodiments, the clasp 322 can include a protrusion 326 configured to engage the recess 226 of the intervertebral cage 200. In some embodiments, the plate 300 can include two clasps 322 and the protrusion 326 of each clasp 322 can be configured to extend substantially towards the other clasp 322. In some embodiments, the protrusion 326 can include an angled surface 324 configured to complement the chamfer 224 of the intervertebral cage 200 and promote the deflection of the clasp 322 during the coupling process. In some embodiments, the protrusion 326 can be configured to block the plate 300 from backing away from the intervertebral cage 200 once in a locked position. In some embodiments, the protrusions 326 of the clasps 322 engage a portion of the recess 226 once the protrusions 326 clear the recess 226 and the clasps 322 return from their deflected position towards their undeflected position and enter a locked position, coupling the plate 300 to the intervertebral cage 200.

In another embodiment (not illustrated), the cage coupling portion 220 of the intervertebral cage 200 can include an internally threaded aperture formed through the anterior face 206 of the intervertebral cage 200 configured to accept a fastener. The plate coupling portion 320 of the plate 300 can include an aperture formed therethrough such that a fastener can be utilized to couple the plate 300 to the intervertebral cage 200. In some embodiments, a portion of the fastener can pass through the aperture of the plate 300 and engage the internally threaded aperture of the intervertebral cage 200. The internally threaded aperture of the intervertebral cage 200 can include an annular protrusion 326 surrounding the internally threaded aperture configured to engage an annular recess 226 formed in the plate 300. The plate 300 can include an annular recess 226 surrounding the aperture of the plate 300, the annular recess 226 configured to receive the annular protrusion 326 of the intervertebral cage 200.

FIG. 6A illustrates a front view of the modular cage system 100 of FIG. 1. FIG. 6B illustrates a side view of the modular cage system 100 of FIG. 1. The anatomy of some patients' spines is prohibitive to utilizing existing intervertebral cage 200 designs and fixation means. For example, in some cases a bone screw 420 may not be the ideal method of fixation due to its shape and configuration. As described herein, bone fixation members 400 can include various methods and instruments for fixation which may include for example, bone screws 420 and bone blades 410. In some embodiments, the modular cage system 100 can include one or more bone screws 420. Bone screws 420, as illustrated in FIGS. 6A and 6B, are generally limited to a straight configuration without any bends or curves. In some embodiments, the modular cage system 100 can include one or more bone blades 410. In some embodiments, bone blades 410 are curved, allowing the bone blade 410 to engage a vertebra in a different manner and orientation than a bone screw 420. In some embodiments, the bone blades 410 can have a straight configuration. In some embodiments, the plate 300 can be configured to optionally receive a plurality of bone screws 420, a plurality of bone blades 410, a combination of bone screws 420 and bone blades 410, or any other combination of bone screws 420, bone blades 410, or other bone fixation members 400. In some embodiments, the first and second receiving portions 330, 340 are each configured to optionally receive either a bone screw 420 or a bone blade 410. This configuration provides options during spinal surgery to utilize the optimal bone fixation member 400 for the patient's anatomy while utilizing the same modular cage system 100. As illustrated in FIGS. 6A and 6B, a combination of bone fixation members 400 can be utilized, which may include for example, a bone blade 410 and a bone screw 420.

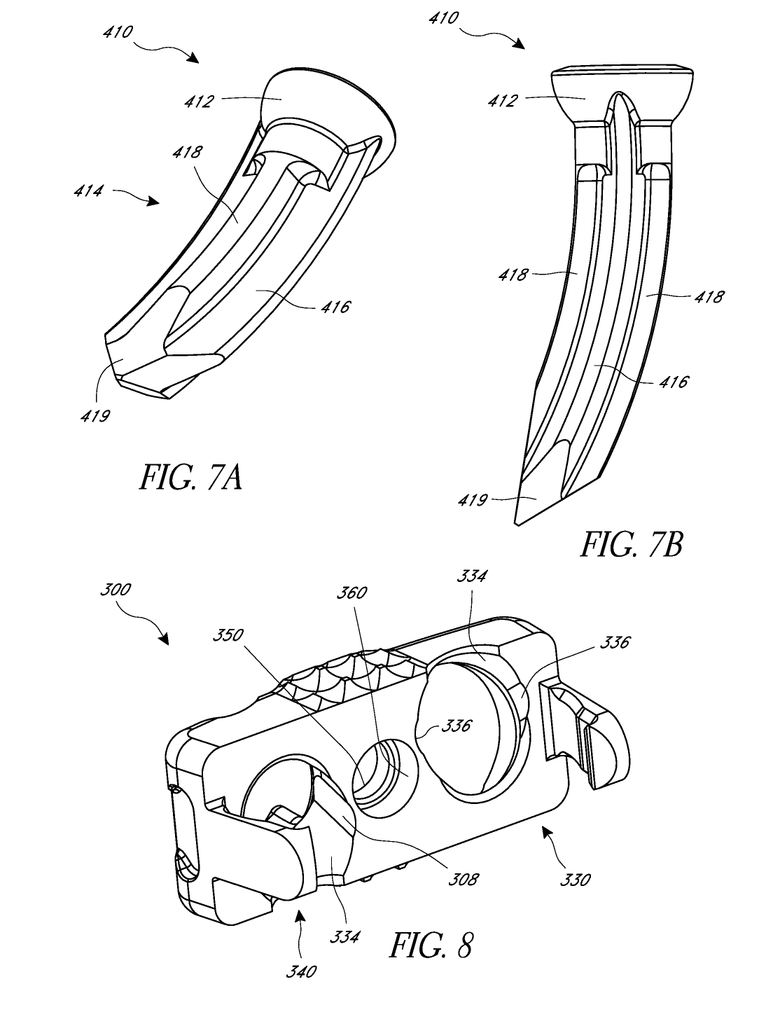

FIG. 7A illustrates a perspective view of one embodiment of a bone blade 410. FIG. 7B illustrates a side view of the bone blade 410 of FIG. 7A. In some embodiments, a bone blade 410 is configured to engage a vertebra via translation without rotation of the bone blade 410. In some embodiments, a bone blade 410 can incorporate a head 412 and a bone engaging portion 414. The head 412 of the bone blade 410 can include a larger diameter than the bone engaging portion 414 of the bone blade 410. The head 412 of the bone blade 410 can be configured to engage the head engaging portion 332 of the plate 300. In some embodiments, the bone engaging portion 414 of the bone blade 410 can include a curve along its central axis as illustrated in FIGS. 7A and 7B. The curve can allow the bone blade 410 to assume a preferred trajectory when engaging a vertebra. The curve can also limit migration of the modular cage system 100 once the bone blade 410 is locked in place as discussed below. The bone engaging portion 414 of the bone blade 410 can include a cross sectional shape configured to translate through bone. In some embodiments, the bone blade 410 can include a major rib 416. In some embodiments, the bone blade 410 can include a minor rib 418. The major and minor rib 418 can include protrusions extending outward from the central axis of the bone blade 410. In some embodiments, the major rib 416 can extend further outward from the central axis of the bone blade 410 then the minor rib 418. In some embodiments, the major rib 416 can extend outward from the central axis of the bone blade 410 substantially the same distance as the minor rib 418. The curve of the bone engaging portion 414 of the bone blade 410 can include a center of curvature. In some embodiments, a minor plane, defined by the center of the minor rib 418, intersects the center of curvature of the curve of the bone engaging portion 414. In some embodiments, the major rib 416 is substantially perpendicular to the minor rib 418 along the curve of the bone engaging portion 414. In some embodiments, the bone engaging portion 414 also includes a tip 419 at the end of the bone engaging portion 414 opposite the head 412. In some embodiments, the tip 419 is configured to pierce a vertebra and aid in the insertion of the bone blade 410 into a vertebra.

FIG. 8 illustrates a perspective view of the intervertebral cage 200 of FIG. 2. In some embodiments, the plate 300 is configured to guide the bone blade 410 into a vertebra. The plate 300 can include features which accept the bone blade 410 in a particular orientation, and ensure the bone blade 410 follows a preferred trajectory into a vertebra. In some embodiments, the plate 300 can include at least one guidance rail 336 configured to accept the major rib 416 of the bone blade 410 and guide the bone blade 410 into a vertebra. In some embodiments, the guidance rails 336 can include recesses formed therein a wall of the through bore 334 of a receiving portion configured to guide the bone blade 410 into a vertebra as the bone blade 410 is passed through the receiving portion. In some embodiments, the guidance rails 336 are arranged substantially parallel to a center axis of the through bore 334. In some embodiments the major rib 416 of the bone blade 410 is configured to engage the guidance rails 336 of the plate 300.

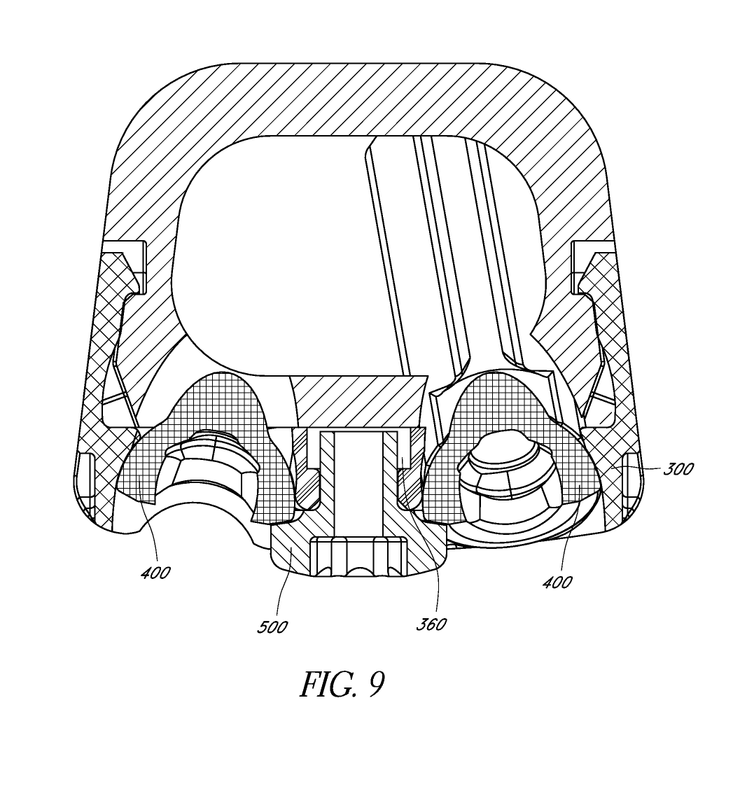

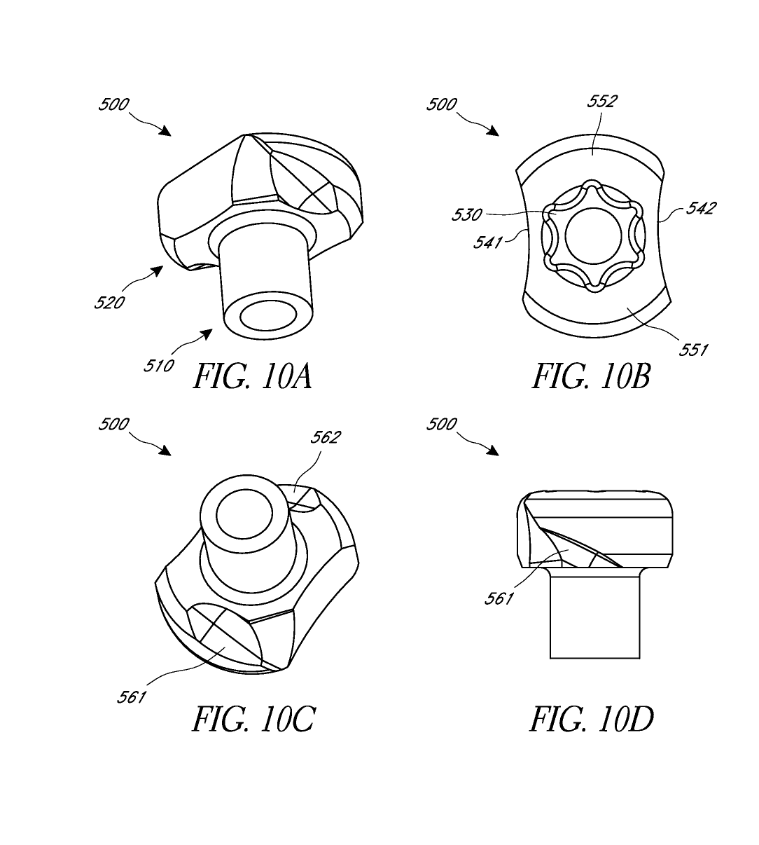

FIG. 9 illustrates a top section view of the plate 300 of FIG. 3 including one embodiment of a retention member 500 in the locked position. FIG. 10A illustrates a perspective view of one embodiment of a retention member 500. FIG. 10B illustrates a top view of the retention member 500 of FIG. 10A. FIG. 10C illustrates a perspective view of the retention member 500 of FIG. 10A. FIG. 10D illustrates a side view of the retention member 500 of FIG. 10A. In some embodiments, the modular cage system 100 can include a retention member 500 configured to block the bone fixation members 400 from backing out of vertebra and the modular cage system 100. In some embodiments, the retention member 500 can lock the bone fixation members 400 in place relative to the plate 300. In some embodiments, the plate 300 includes a central bore (See also FIG. 8) configured to accept the retention member 500. In some embodiments, the retention member 500 can include a shaft portion 510 and a blocking portion 520. As illustrated in FIG. 9, the shaft portion 510 can be configured to engage and couple the retention member 500 to the plate 300. The blocking portion 520 can be configured to engage the first and second bone fixation members 400. In some embodiments the blocking portion 520 can include a tool recess 530 configured to accept a tool configured to rotate the retention member 500 between an unlocked position and a locked position. In an unlocked position, the bone fixation members 400 can be installed through the receiving portions of the plate 300. In a locked position, the bone fixation members 400 are locked in place. In some embodiments, the retention member 500 is configured to lock two bone fixation members 400 in place. In some embodiments, the retention member 500 is configured to rotate approximately 90 degrees to alternate between an unlocked and a locked position.

In some embodiments, the blocking portion 520 can include first and second clearance sides 541, 542 and first and second interference sides 551, 552. In some embodiments, the first and second clearance sides 541, 542 each comprise a recess formed therein the blocking portion 520 of the retention member 500 providing room for the bone fixation members 400 to enter the receiving portions of the plate 300. In some embodiments, the first and second interference sides 551, 552 include first and second engaging portions 561, 562 respectively. The first engaging portion 561 can be configured to engage a first bone fixation member 400 angled in a first direction. The second engaging portion 562 can be configured to engage a second bone fixation member 400 angled in a different direction. The first and second engagement portions can be configured to engage the bone fixation members 400 when the retention member 500 is rotated from an unlocked position to a locked position. In some embodiments, the retention member 500 is configured such that an interference fit exists between the blocking portion 520 of the retention member 500 and the heads 412 of the first and second bone fixation members 400 when the retention member 500 is in a locked position. In some embodiments, the interference fit can lock the bone fixation members 400 in place. In some embodiments, the retention member 500 can limit the bone fixation members 400 from rotating. In some embodiments, the retention member 500 can limit the angle of each bone fixation member 400 from changing.

In some embodiments, the retention member 500 can be permanently coupled to the plate 300. In some embodiments, the retention member 500 can be removably coupled to the plate 300. In some embodiments, the plate 300 can include a posterior bore 360 which can have a larger diameter than the central bore. The shaft portion 510 of the retention member 500 can be configured to be enlarged or have an enlarged portion affixed to the end opposite the blocking portion 520 such that the enlarged portion of the shaft portion 510 protrudes into the posterior bore 360, blocking the retention means from backing out of the central bore and the plate 300. In some embodiments, the retention member 500 can be installed on the plate 300 prior to implantation of the plate 300. In some embodiments, the retention member 500 can be installed on the plate 300 after implantation of the plate 300. In some embodiments, the retention member 500 can be temporarily coupled to the plate 300. In some embodiments, the retention member 500 can be retained in the plate 300 via a snap fit configuration. In some embodiments, the retention member 500 can be coupled to the plate 300 via a threaded shaft portion 510 and internally threaded central bore. In some embodiments, the retention member 500 can include an aperture formed therethrough for passage of a fastener configured to engage the plate 300 and retain the retention member 500.

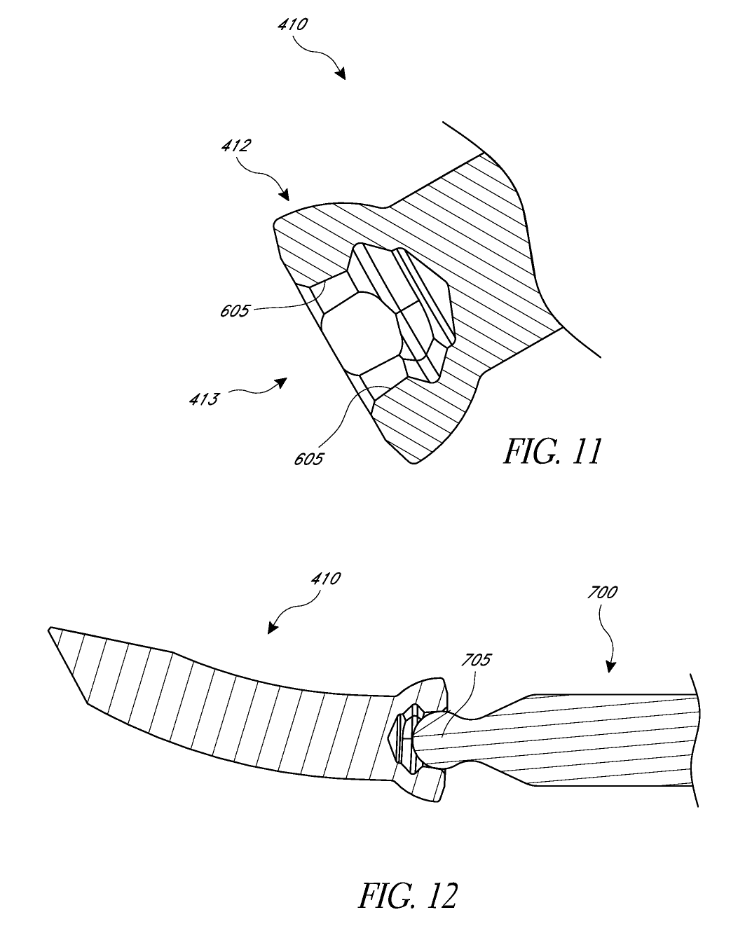

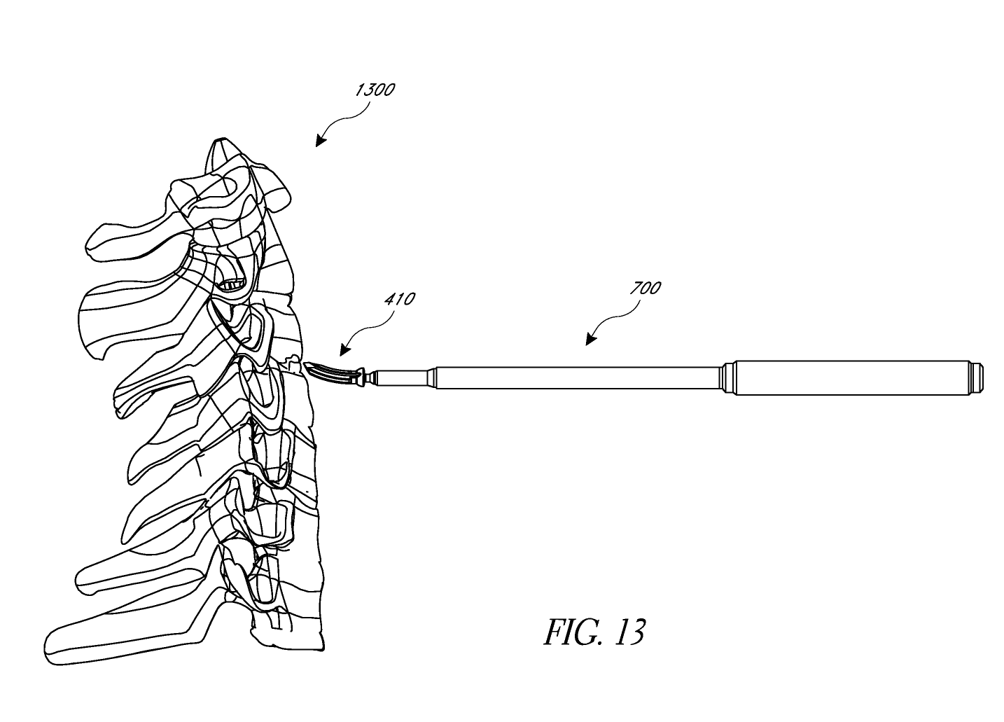

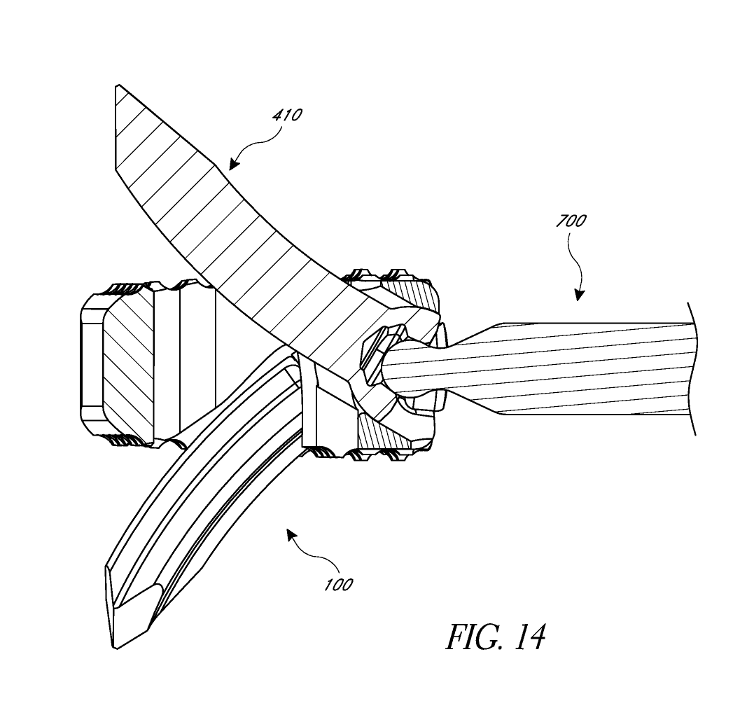

FIG. 11 illustrates a partial section view of the bone blade 410 of FIG. 7A. FIG. 12 illustrates a partial section view of one embodiment of a bone blade insertion tool 700 engaged to the bone blade 410 of FIG. 7A. FIG. 13 illustrates a partial section view of the bone blade insertion tool 700 of FIG. 12 engaged to the bone blade 410 of FIG. 7A and approaching the delivery site. FIG. 14 illustrates a partial section view of the bone blade 410 of FIG. 7A fully installed in the plate 300 and engaged by the bone blade insertion tool 700 of FIG. 12. In some embodiments, the head 412 of the bone blade 410 can include a tool receiving portion 413. In some embodiments, the modular cage system 100 can include a bone blade insertion tool 700. In some embodiments, the tool receiving portion 413 can be configured to receive a bone blade insertion tool 700. In some embodiments, the bone blade insertion tool 700 can include an engaging element 705 configured to engage the tool receiving portion 413 of the bone blade 410. In some embodiments, the engaging element 705 can be spherical. In some embodiments, the tool receiving portion 413 can include a taper 605 configured to accept the engaging element 705 of the bone blade insertion tool, such that the bone blade insertion tool 700 can apply a force substantially in perpendicular to the spinal column 1300 and parallel with the central axis of the bone engaging portion 414 adjacent the head 412 of the bone blade 410. The taper 605 can provide for a locking arrangement with the engaging element 705 of the bone blade insertion tool 700 such that the bone blade insertion tool can deliver the bone blade 410 to the implantation site and force the bone blade 410 through the modular cage system 100 and into the vertebra, but allow the bone blade 410 to unlock from the bone blade insertion tool 700 and angle relative to the bone blade insertion tool 700 during installation as the bone blade 410 follows its curved trajectory into the vertebra, and still allow the bone blade insertion tool 700 to transfer force through the head 412 of the bone blade 410, forcing the bone blade 410 into the vertebra.

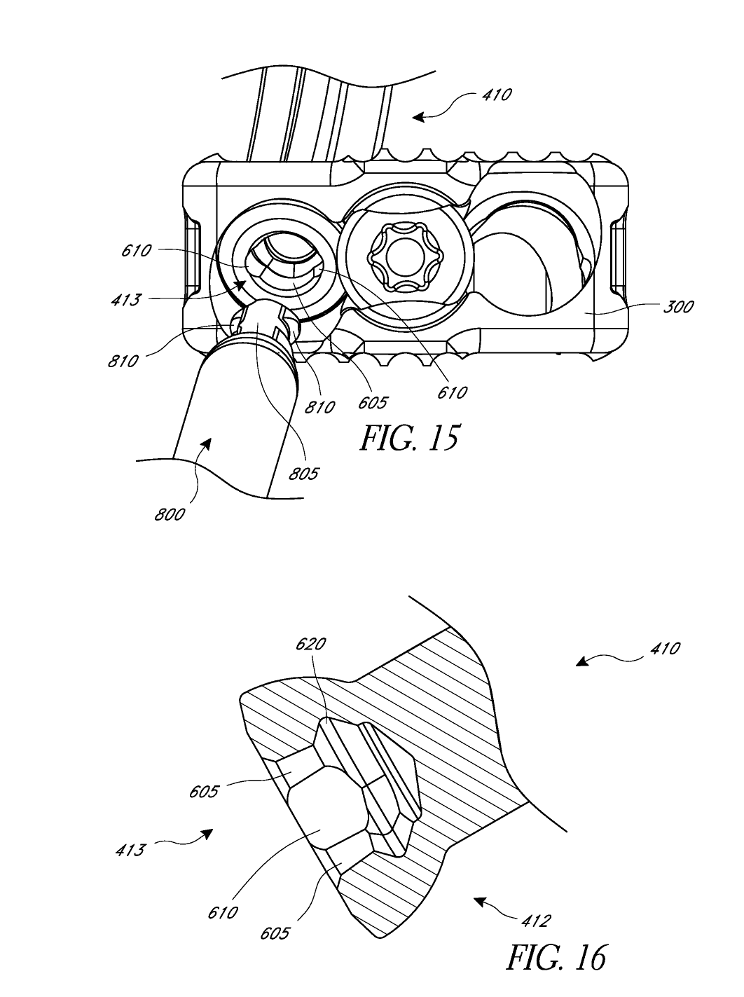

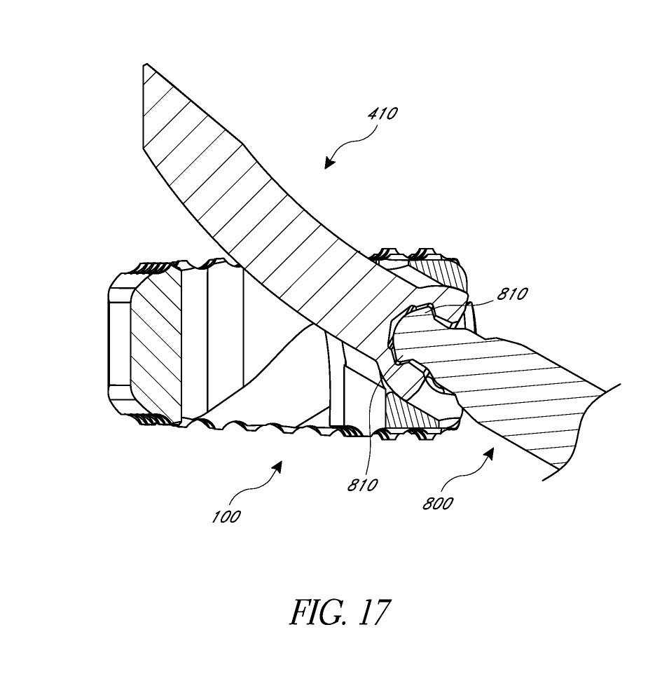

FIG. 15 illustrates a front view of the modular cage system 100 of FIG. 1 including one embodiment of a bone blade extraction tool 800. FIG. 16 illustrates a partial section view of the bone blade 410 of FIG. 7A. FIG. 17 illustrates a partial section view of the bone blade 410 of FIG. 7A fully installed in the plate 300 and engaged by the bone blade extraction tool 800 of FIG. 15. In some embodiments, the modular cage system 100 can include a bone blade extraction tool 800. In some embodiments, the tool receiving portion 413 can be configured to receive the bone blade extraction tool 800. In some embodiments, the bone blade extraction tool 800 can include an extension portion 805 configured to extend into the tool receiving portion 413 of the bone blade 410. In some embodiments, at least a portion of the extension portion 805 can be substantially cylindrical in shape. In some embodiments, the extension portion 805 can be configured to slide within the taper 605 of the tool receiving portion 413 of the bone blade 410.

In some embodiments, the extension portion 805 can include at least one extraction lobe 810 protruding radially outwards from the exterior of the extension portion 805. In some embodiments, the tool receiving portion 413 can include at least one lobe recess 610 formed in the taper 605 of the tool receiving portion 413 of the bone blade 410. In some embodiments, the at least one lobe recess 610 can be formed in a non-tapered portion of the bone blade 410 (not illustrated). In some embodiments, the extraction lobes can be configured such that the extraction lobes 810 and extension portion 805 can only be inserted into the tool receiving portion 413 when the bone blade extraction tool 800 is rotated such that the extraction lobes 810 line up with the lobe recesses 610 in the tool receiving portion 413 of the bone blade 410. In some embodiments, the tool receiving portion 413 of the bone blade 410 can include an undercut channel 620 configured to rotatably accept the extraction lobes 810 of the bone blade extraction tool 800. In some embodiments, the bone blade extraction tool 800 can be rotated such that the extraction lobes 810 align with the lobe recesses 610, inserted into the tool receiving portion 413 of the bone blade 410 until the extraction lobes 810 reach the undercut channel 620, rotated such that the extraction lobes 810 do not align with the lobe recesses 610, and pulled away from the modular cage system 100, pulling the bone blade 410 out of the modular cage system 100.

In some embodiments, the bone blade extraction tool 800 can be rotated from an aligned position, as illustrated in FIG. 15, wherein the extraction lobes 810 are aligned with the lobe recesses 610, to an unaligned position, as illustrated in FIG. 17, wherein the extraction lobes 810 do not align with the lobe recesses 610, once the extraction lobes 810 are inserted into the undercut channel 620 of the tool receiving portion 413. The bone blade extraction tool 800 can then exert a force through the extraction lobes 810, removing the bone blade 410 from the modular cage system 100. In some embodiments, the undercut channel 620 can be configured to lock the bone extraction tool 800 to the bone blade 410 when the bone blade extraction tool 800 is rotated from an aligned position to an unaligned position. In some embodiments, the undercut channel 620 can include an outer radius which decreases as the bone blade extraction tool 800 is rotated from an aligned position to an unaligned position. In some embodiments, the decrease in radius can cause an interference fit between the undercut channel 620 and the extraction lobes 810, locking the bone blade extraction tool 800 to the bone blade 410.

In some embodiments, the thickness of the undercut channel 620 can decrease as the bone extraction tool 800 is rotated from an aligned position to an unaligned position. In some embodiments, the decrease in thickness can cause an interference fit between the undercut channel 620 and the extraction lobes 810, locking the bone blade extraction tool 800 to the bone blade 410. In some embodiments, such a locking feature can limit the bone blade 410 from detaching from the bone blade extraction tool 800 during removal. In some embodiments, rotation from an aligned position to an unaligned position can require between approximately 45 and 135 degrees of rotation. In some embodiments, the rotation could be larger than this range. In some embodiments, the rotation could be smaller than this range. In some embodiments, rotation from an aligned position to an unaligned position can require between approximately 75 and 105 degrees of rotation. In some embodiments, rotation from an aligned position to an unaligned position can require approximately 90 degrees of rotation.

Various modifications to the implementations described in this disclosure may be readily apparent to those skilled in the art, and the principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein. Additionally, a person having ordinary skill in the art will readily appreciate, the terms “upper” and “lower” are sometimes used for ease of describing the figures, and indicate relative positions corresponding to the orientation of the figure on a properly oriented page, and may not reflect the proper orientation of the device as implemented.

Certain features that are described in this specification in the context of separate implementations also can be implemented in combination in a single implementation. Conversely, various features that are described in the context of a single implementation also can be implemented in multiple implementations separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes. However, other operations that are not depicted can be incorporated in the example processes that are schematically illustrated. For example, one or more additional operations can be performed before, after, simultaneously, or between any of the illustrated operations. In certain circumstances, multitasking and parallel processing may be advantageous. Moreover, the separation of various system components in the implementations described above should not be understood as requiring such separation in all implementations. Additionally, other implementations are within the scope of the following claims. In some cases, the actions recited in the claims can be performed in a different order and still achieve desirable results.

In describing the present technology, the following terminology may have been used: The singular forms “a,” “an,” and “the” include plural referents unless the context clearly dictates otherwise. Thus, for example, reference to an item includes reference to one or more items. The term “ones” refers to one, two, or more, and generally applies to the selection of some or all of a quantity. The term “plurality” refers to two or more of an item. The term “about” means quantities, dimensions, sizes, formulations, parameters, shapes and other characteristics need not be exact, but may be approximated and/or larger or smaller, as desired, reflecting acceptable tolerances, conversion factors, rounding off, measurement error and the like and other factors known to those of skill in the art. The term “substantially” means that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide. Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3 and 4 and sub-ranges such as 1-3, 2-4 and 3-5, etc. This same principle applies to ranges reciting only one numerical value (e.g., “greater than about 1”) and should apply regardless of the breadth of the range or the characteristics being described. A plurality of items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. Furthermore, where the terms “and” and “or” are used in conjunction with a list of items, they are to be interpreted broadly, in that any one or more of the listed items may be used alone or in combination with other listed items. The term “alternatively” refers to selection of one of two or more alternatives, and is not intended to limit the selection to only those listed alternatives or to only one of the listed alternatives at a time, unless the context clearly indicates otherwise.

It should be noted that various changes and modifications to the presently preferred embodiments described herein will be apparent to those skilled in the art. Such changes and modifications may be made without departing from the spirit and scope of the invention and without diminishing its attendant advantages. For instance, various components may be repositioned as desired. It is therefore intended that such changes and modifications be included within the scope of the invention. Moreover, not all of the features, aspects and advantages are necessarily required to practice the present invention. Accordingly, the scope of the present invention is intended to be defined only by the claims that follow.

What is claimed is:

1. A spinal implant comprising:
a plate configured to couple to an intervertebral cage, the intervertebral cage configured and arranged to be positioned in an intervertebral space between a first vertebra and a second vertebra, wherein the plate comprises an upper surface configured to engage the first vertebra and a lower surface opposite the upper surface, the lower surface configured to engage the second vertebra, wherein the plate comprises a posterior face configured to face the intervertebral space and abut an anterior face of the intervertebral cage and an anterior face configured to face outward from the intervertebral space;
wherein the plate comprises a first receiving portion and a second receiving portion, the first receiving portion angled relative to the second receiving portion, the first receiving portion configured to receive a first bone fixation member, the first bone fixation member comprising either a bone screw or a bone blade, the first bone fixation member configured to engage a first vertebra, the second receiving portion configured to receive a second bone fixation member, the second bone fixation member comprising either a bone screw or a bone blade, the second bone fixation member configured to engage a second vertebra, the first and second receiving portions each configured to optionally receive either a bone screw or a bone blade.

2. The spinal implant of claim 1, wherein the first and second receiving portions each comprise at least one guidance rail configured to guide a bone blade into a vertebra.

3. The spinal implant of claim 1, wherein the first and second receiving portions each comprise a through bore and a head engaging portion, each through bore configured to receive the bone engaging portion of the bone fixation member, each head engaging portion configured to block the head of the bone fixation member from passing completely through the receiving portion.

4. The spinal implant of claim 3, wherein the through bore of the first and second receiving portions each comprise at least one guidance rail, wherein each guidance rail comprises a recess formed therein a wall of each through bore, each guidance rail configured to accept a portion of the bone blade and guide the bone blade into a vertebra as it is passed through the receiving portion.

5. The spinal implant of claim 4, wherein the each receiving portion is configured to receive a bone blade, wherein the bone blade comprises a head and a bone engaging portion, wherein the bone engaging portion of the bone blade comprises a curve along its length, wherein the bone engaging portion of the bone blade comprises a major rib, wherein the bone blade is configured to engage a vertebra via translation without rotation of the bone blade, wherein the each guidance rail is configured to accept the major rib of the bone blade and guide the bone blade into a vertebra.

6. The spinal implant of claim 5, wherein the bone engaging portion of the bone blade comprises a minor rib arranged perpendicular to the major rib.

7. The spinal implant of claim 6, wherein each through bore of each receiving portion comprises a central axis, and wherein each of the guidance rails are arranged substantially parallel to a central axis of the through bore.

8. A spinal implant system comprising:
an intervertebral cage configured and arranged to be positioned in an intervertebral space between a first vertebra and a second vertebra, the intervertebral cage comprising an upper surface and a lower surface opposite the upper surface, the intervertebral cage comprising a posterior face and an anterior face opposite said posterior face, the intervertebral cage comprising at least two sidewalls;
a plate configured to couple to the intervertebral cage, the intervertebral cage configured and arranged to be positioned in the intervertebral space, wherein the plate comprises an upper surface configured to engage the first vertebra and a lower surface opposite the upper surface, the lower surface configured to engage a second vertebra, wherein the plate comprises a posterior face configured to face the intervertebral space and abut an anterior face of the intervertebral cage and an anterior face configured to face outward from the intervertebral space;
at least two bone fixation members adapted for insertion through the receiving portions of the plate and configured for anchoring the plate in an intervertebral space between two vertebrae of a patient, the bone fixation members comprising at least a first bone fixation member and a second bone fixation member, the first bone fixation member comprising either a bone screw or a bone blade and the second bone fixation member comprising either a bone screw or a bone blade, the first bone fixation member configured to engage the first vertebra, the second bone fixation member configured to engage the second vertebra;
wherein the intervertebral cage comprises a first cage bore formed therethrough the anterior face and the upper surface configured to receive the first bone fixation member and a second cage bore formed therethrough the anterior face and the lower surface configured to receive the second bone fixation member;
wherein the plate comprises a first receiving portion and a second receiving portion, the first receiving portion angled relative to the second receiving portion, the first receiving portion configured to receive the first bone fixation member, the second receiving portion configured to receive a second bone fixation member, the first and second receiving portions each configured to optionally receive either a bone screw or a bone blade;
wherein the plate comprises a plate coupling portion configured to couple the plate to the intervertebral cage; and
wherein the intervertebral cage comprises a cage coupling portion configured to couple the cage to the plate.

9. The spinal implant system of claim 8, wherein the intervertebral cage is constructed of PEEK and the plate is constructed of titanium.

10. The spinal implant system of claim 8, further comprising:
a retention member coupled to the plate, the retention member configured to block first and second bone fixation members from backing out of the plate;
wherein the retention member comprises a shaft portion and a blocking portion, the shaft portion configured to engage and couple the retention member to the plate, the blocking portion configured to engage the first and second bone fixation members;
wherein the retention member is configured to rotate between a locked position and an unlocked positions such that when in an unlocked position, the first and second bone fixation members can be installed through the first and second receiving portions of the plate, and in a locked position, the first and second bone fixation members are locked in place.

11. The spinal implant system of claim 10, wherein the blocking portion comprises at least a first clearance side and a first interference side, and a second clearance side and a second interference side, wherein the first and second clearance sides each comprise a recess formed therein the blocking portion of the retention member, providing room for the first and second bone fixation members to enter the first and second receiving portions of the plate, wherein the first and second interference sides include first and second engaging portions respectively, the first engaging portion configured to engage the head of the first bone fixation member and the second engaging portion configured to engage the head of the second bone fixation member when the retention member is rotated from an unlocked position to a locked position.

12. The spinal implant system of claim 11, wherein the retention member is configured such that interference fit exists between the blocking portion of the retention member and the heads of the first and second bone fixation members when the retention member is in a locked position.

13. The spinal implant system of claim 8, wherein the first and second receiving portions each comprise at least one guidance rail configured to guide a bone fixation member comprising a bone blade into a vertebra, the first and second receiving portions each comprise a through bore and a head engaging portion, each through bore configured to receive the bone engaging portion of the bone fixation member, each head engaging portion configured to block the head of the bone fixation member from passing completely through the receiving portion, wherein the through bore of the first and second receiving portions each comprise at least one guidance rail, wherein each guidance rail comprises a recess formed therein a wall of each through bore, each guidance rail configured to accept a portion of the bone blade and guide the bone blade into a vertebra as it is passed through the receiving portion, each receiving portion is configured to receive a bone blade, wherein the bone blade comprises a head and a bone engaging portion, wherein the bone engaging portion of the bone blade comprises a curve along its length, wherein the bone engaging portion of the bone blade comprises a major rib, wherein the bone blade is configured to engage a vertebra via translation without rotation of the bone blade, wherein the each guidance rail is configured to accept the major rib of the bone blade and guide the bone blade into a vertebra, and wherein each through bore of each receiving portion comprises a central axis, and wherein each of the guidance rails are arranged substantially parallel to the central axis of the through bore.

14. The spinal implant system of claim 8, wherein the cage coupling portion of the intervertebral cage comprises at least one channel configured to receive a portion of the plate, and wherein the plate coupling portion of the plate comprises at least one clasp extending outwards from the posterior face of the plate, the at least one clasp configured to enter the channel of the intervertebral cage and couple the plate to the intervertebral cage.

15. The spinal implant system of claim 8, wherein the intervertebral cage comprises at least one chamfer configured to deflect the at least one clasp, wherein the intervertebral cage comprises at least one recess formed in the at least one channel, the recess configured to receive a portion of the at least one clasp, wherein the at least one clasp comprises a protrusion configured to engage the recess of the intervertebral cage, and wherein the at least one clasp is configured to deflect when coupling the intervertebral cage and to return toward the undeflected position when the protrusion engages the recess of the intervertebral cage and couples the plate to the intervertebral cage.

16. The spinal implant system of claim 8, wherein the plate comprises a plate height defined by the distance between the upper surface and the lower surface of the plate, wherein the intervertebral cage comprises an anterior cage height defined by the distance between the upper surface and the lower surface of the cage measured at an anterior face of the intervertebral cage, wherein the plate height is substantially the same as the anterior cage height.

17. A method for installing a spinal implant system comprising:
positioning an intervertebral cage in an intervertebral space between a first vertebra and a second vertebra;
positioning a plate at least partially in the intervertebral space and coupling the plate to the intervertebral cage;
wherein coupling the plate to the intervertebral cage comprises forcing the plate against the anterior face of the intervertebral cage;
installing a first bone fixation element through a first receiving portion of the plate and into a first vertebra, the first vertebra located above the intervertebral cage;
installing a second bone fixation element through a first receiving portion of the plate and into a second vertebra, the second vertebra located below the intervertebral cage;
wherein at least one of the first bone fixation element and second bone fixation element comprises a bone blade;
rotating a retention member 90 degrees to lock the first and second bone fixation elements in place.

18. The method of claim 17, wherein installation of the bone blade comprises aligning a major rib of the bone blade in a guidance rail located in the first or second receiving portion and exerting a force substantially perpendicular a center axis of the spinal column in the posterior direction causing the bone blade to enter the first or second vertebra.

19. The method of claim 18, wherein the bone blade comprises a curve along a bone engaging portion of the bone blade.

20. The method of claim 17, wherein rotating a retention member comprises creating an interference fit between the retention member and the first and second bone fixation elements, locking the first and second bone fixation elements in place.

21. A method of removing a bone blade which is implanted into an intervertebral cage and a vertebra comprising:
maneuvering a bone blade extraction tool towards the bone blade;
rotating the bone blade extraction tool until at least one extraction lobe of the bone blade extraction tool aligns with at least one lobe recess of a tool receiving portion of the bone blade;
inserting the bone blade extraction tool into the tool receiving portion of the bone blade until the at least one extraction lobe reaches an undercut channel of the tool receiving portion;
rotating the bone blade extraction tool until the at least one extraction lobe locks the bone blade extraction tool to the bone blade; and
pulling the bone blade extraction tool away from the intervertebral cage.

22. The method of claim 21, wherein the at least one extraction lobe locking the bone blade extraction tool to the bone blade comprises the at least one extraction lobe achieving an interference fit between the at least one extraction lobe and the undercut channel.

23. The method of claim 21, wherein the undercut channel comprises a decreasing diameter configured to achieve an interference fit with the at least one extraction lobe of the bone blade extraction tool.
